(19) United States
(12) Patent Application Publication
(10) Pub. No.: US 2025/0295525 A1
(43) Pub. Date: Sep. 25, 2025

(54) MACULAR HOLE MEASUREMENT FOR HANDHELD INSTRUMENTS

(71) Applicant: Alcon Inc., Fribourg (CH)

(72) Inventors: Niccolo Maschio, Winterthur (CH); Niels Alexander Abt, Winterthur (CH); Reto Grüebler, Greifensee (CH)

(21) Appl. No.: 19/057,045

(22) Filed: Feb. 19, 2025

Related U.S. Application Data
(60) Provisional application No. 63/567,656, filed on Mar. 20, 2024.

(51) Int. Cl. A61F 9/007; A61B 17/29

(52) U.S. Cl. CPC A61F 9/007; A61B 17/2909

(57) ABSTRACT

An ophthalmic surgical instrument for measuring tissues is provided. The instrument includes a handle comprising a distal end and a proximal end, an instrument portion extending from the distal end of the handle, and an outer tube located around the instrument portion. The instrument portion of the surgical instrument further includes a distal end comprising an end effector. The degree of movement of the end effector corresponds to a scale on the handle of the surgical instrument that facilitates the measurement of a tissue during ophthalmic procedures. The movement of the outer tube or the end effector may further facilitate measurement of the tissue.

### INTRODUCTION

The human eye can suffer a number of maladies causing mild deterioration to complete loss of vision. While contact lenses and eyeglasses can compensate for some ailments, ophthalmic surgery may be required for others. Generally, ophthalmic surgery may be classified into posterior segment procedures, such as vitreoretinal surgery, and anterior segment procedures, such as cataract surgery. Vitreoretinal surgery may address many different eye conditions, including, but not limited to, macular degeneration, diabetic retinopathy, diabetic vitreous hemorrhage, macular hole, detached retina, and epiretinal membrane.

During surgery to treat macular holes, a surgical procedure may require incisions and insertion of tools within an eye to either peel the internal limiting membrane (ILM) or create a flap of the ILM to cover the macular hole. The ILM is a thin transparent membrane positioned between the vitreous and the retina of the eye, which may pull on the retina and cause various eye maladies, including macular holes. When a user is suffering from a macular hole, depending on the size of the macular hole, a surgical procedure may be selected from peeling the ILM or creating an ILM flap. In cases where the macular hole is less than a specified size, a surgeon may choose to perform a peeling of the ILM to prevent further damage to the retina (e.g., widening of the macular hole) and the macular hole may be expected to close over time in the absence of the ILM. In certain other cases where the macular hole is larger than a specified size, a surgeon may choose to create an ILM flap, which requires lifting a small portion of the ILM and placing the portion on top of, or within, the macular hole.

### SUMMARY

Aspects of the present disclosure relate to a method of measurement for use in surgical settings, and more specifically, measurement of macular holes using a surgical instrument.

Certain embodiments of the present disclosure provide an ophthalmic surgical instrument including a handle, including a distal end and a proximal end, and an instrument portion extending from the distal end of the handle, the instrument portion comprising a distal end. The distal end of the instrument portion includes an end effector and a degree of movement of the end effector of the instrument corresponds to a scale on the handle of the surgical instrument that facilitates the measurement of a tissue during ophthalmic procedures. The ophthalmic surgical instrument further includes an outer tube located around the instrument portion, and the movement of the outer tube or the end effector facilitates measurement of the tissue.

Certain embodiments of the present disclosure provide an ophthalmic surgical instrument including a handle, including a distal end and a proximal end, and an instrument portion extending from the distal end of the handle, the instrument portion including a distal end. The distal end of the instrument portion includes an end effector and the end effector has a measurement scale thereon corresponding to a sizing scale for facilitating measurement of a tissue during ophthalmic procedures.

The following description and the related drawings set forth herein detail certain illustrative features of one or more embodiments, including those described above.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a perspective view of an exemplary surgical instrument having a loop, according to embodiments described herein.

FIG. 2 illustrates a perspective view of an exemplary surgical instrument within an eye during an ophthalmic surgical procedure, according to embodiments described herein.

FIG. 3A illustrates a perspective view of an exemplary surgical instrument having forceps, according to embodiments described herein.

FIG. 3B illustrates a perspective view of an exemplary forceps that may be implemented in the surgical instrument of FIG. 3A, according to embodiments described herein.

FIG. 4A illustrates a perspective view of an exemplary surgical instrument having a retractable soft tip in an extended position, according to embodiments described herein.

FIG. 4B illustrates a perspective view of an exemplary surgical instrument having a retractable soft tip in a retracted position, according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to a method of measurement for use in surgical settings, and more specifically, measurement of macular holes using a surgical instrument.

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end, segment, or portion of a component refers to the end, segment, or portion that is closer to a patient's body during use thereof. On the other hand, a proximal end, segment, or portion of the component refers to the end, segment, or portion that is distanced further away from the patient's body and is in proximity to, for example, a surgical laser system.

As described herein, during a surgical procedure to repair a macular hole, a surgeon may begin the procedure by peeling a portion of the ILM. Depending on the size of the macular hole, the surgeon may then proceed to peeling the remainder of the ILM away from the retina or pulling the flap created by peeling the ILM over the macular hole. However, a surgeon may have difficulty determining the size of the macular hole, which can result in selecting the incorrect procedure, eventually leading to incorrect healing of the macular hole and/or additional surgical procedures to treat the macular hole. Further, existing methods for determining the size of the macular hole require imaging before the surgical procedure and/or the use of an additional surgical instrument (e.g., an optical coherence tomography (OCT) device) in order to determine the size of the hole. Existing methods are infrequently used due to the cost and inconvenience of running additional imaging tests or using an additional imaging device during a procedure. As such, current methods of determining the size of a macular hole and, therefore, for assisting the surgeon in quickly and efficiently choosing between performing an ILM peeling procedure or an ILM flap procedure present a variety of limitations.

Accordingly, the surgical instruments described herein overcome many of the limitations associated with current methods used for measuring the size of a macular hole.

Certain embodiments of the present disclosure provide improved surgical instrument for use in ophthalmic procedures. More particularly, certain embodiments provide surgical instruments that provide an accurate measurement of macular hole size without additional intra-operative devices and/or pre-operative imaging required. Such surgical instruments may be configured to perform a certain function during the vitrectomy and provide a measurement of an ophthalmic tissue (e.g., a macular hole). For example, a surgical instrument may be configured to peel the ILM and provide a measurement of the macular hole. In another example, a surgical instrument may be configured to aspirate fluid and/or material from the eye and provide measurement of an ophthalmic tissue (e.g., a macular hole).

Certain embodiments of the present disclosure are directed to an ophthalmic surgical instrument for measuring an ophthalmic tissue (e.g., a macular hole). The ophthalmic surgical instrument includes a handle, an instrument portion extending from the distal end of the handle, and an outer tube located around the instrument portion, wherein movement of the outer tube or an end effector facilitates measurement of a tissue. Further, the distal end of the instrument portion includes the end effector, and a degree of movement of the end effector of the instrument corresponds to a scale on the handle of the surgical instrument that facilitates measurement of the tissue during ophthalmic procedures.

Certain other embodiments of the present disclosure are directed to an ophthalmic surgical instrument including a handle, an instrument portion extending from the distal end of the handle, and an outer tube located around the instrument portion. The instrument portion comprises a distal end including an end effector and the end effector includes a measurement scale thereon corresponding to a sizing scale for facilitating the measurement of a tissue during ophthalmic procedures.

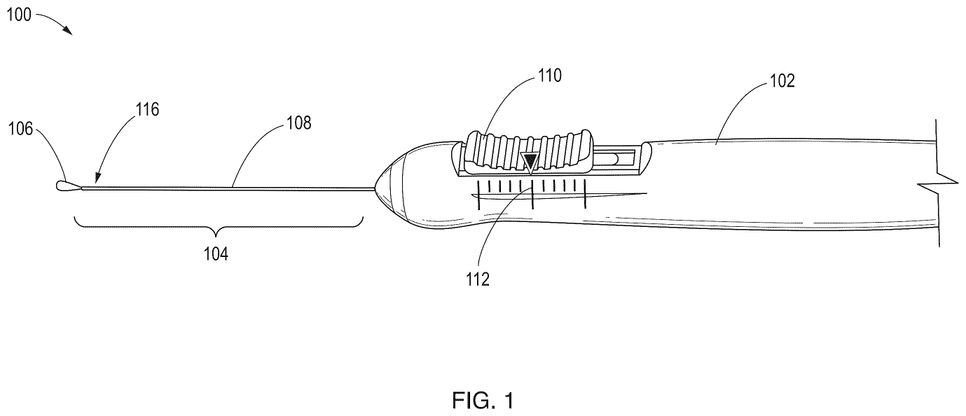

FIG. 1 illustrates a perspective view of an exemplary surgical instrument 100 having an end effector, such as a loop 106, according to embodiments described herein. The surgical instrument 100 includes a handle 102, an instrument portion 104, including a loop 106, an outer tube 108, and an actuator, such as a slider 110. The handle 102 is sized and contoured to be grasped by a hand of a surgeon performing an ophthalmic surgical procedure, such as an ILM peeling or ILM flap procedure. The instrument portion 104 extends from the distal end of the handle 102 and includes a loop 106 at the distal end. The slider 110 may be a manual control structure mounted to the handle 102, configured to translate the loop 106 out of the distal end of the outer tube 108.

The loop 106 may be made of a highly flexible material, such as nitinol (a nickel titanium alloy), spring steel, or other material. The high flexibility enables the loop 106 to elastically deform in order to fit within the outer tube 108 and, when extended from the outer tube 108, expand to a size that is much wider than the outer diameter of the outer tube 108. For example, the loop 106 may expand to a size that is at least two times, four times, eight times, or at least 16 times the outer diameter of the outer tube 108. In certain embodiments, the loop 106 may expand to a diameter of up to 500 μm (micrometers), as described herein. Outer tube 108 may be any suitable medical grade tubing such as titanium, stainless steel, or suitable polymer and is sized so that the loop 106 may fit easily within.

In certain embodiments, the movement of the loop 106 facilitates measurement of the ophthalmic tissue during an ophthalmic procedure. For example, the degree of movement of the loop 106 corresponds to a scale 112 positioned on the handle 102 below the slider 110. The surgeon may release a portion of the loop 106 out of the distal end 116 of the outer tube 108 by manually sliding the slider 110 on the handle 102. The surgeon may continue to expand the loop 106 until the loop 106 corresponds to the size of the macular hole in the patient's retina. The surgeon may alter the positioning of the surgical instrument 100 such that the loop 106 is positioned over the macular hole. Once the diameter of the loop 106 matches the diameter of the macular hole, the position of the slider 110 relative to the scale 112 on the handle 102 may demonstrate the diameter of the macular hole.

The scale 112 on the handle 102 may include markings every 100 μm, such as markings at 0 μm, 100 μm, 200 μm, 300 μm, 400 μm, and 500 μm. Alternatively, the scale 112 may include markings every 50 μm, such as markings at 0 μm, 50 μm, 100 μm, 150 μm, 200 μm, 250 μm, 300 μm, 350 μm, 400 μm, 450 μm, and 500 μm, In certain other embodiments, the scale 112 may include markings at 0 μm and 400 μm. In certain embodiments, the markings may be formed of a removable adhesive material applied to the handle 102 or one or more markings etched within in the surface of the handle 102, for example. If, when the diameter of the loop 106 matches the diameter of the macular hole, the center of the slider 110 is above the 300 μm marking, the surgeon may determine that the macular hole is 300 μm. In certain embodiments, the surgeon may determine whether to complete an ILM peeling procedure or an ILM flap procedure based on the size of the macular hole (e.g., whether the macular hole is 400 μm or greater).

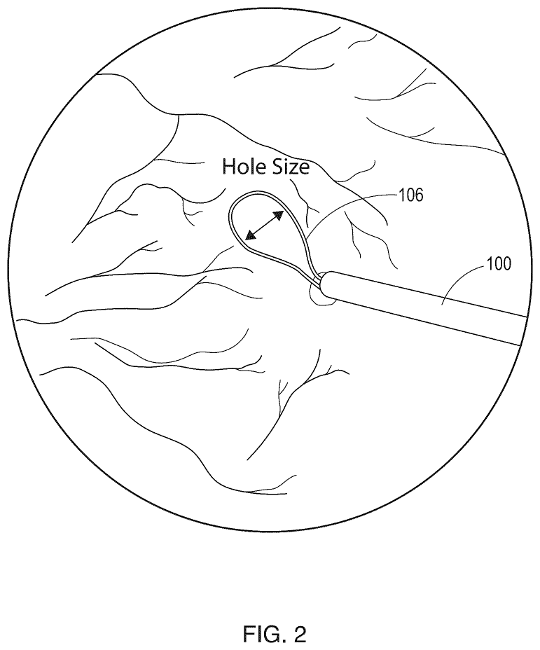

As shown in FIG. 2, the loop 106 is positioned above the macular hole within the eye. Once the surgeon enters the eye with the surgical instrument 100, the surgeon may begin to peel the ILM using the loop 106, for example, before positioning the loop 106 over the macular hole to determine the size of the macular hole. If the size of the macular hole is less than 400 μm according to the slider 110 when the loop 106 is the same size as the macular hole, the surgeon may proceed with an ILM peeling procedure to allow the macular hole to close and resolve over time. Alternatively, if the size of the macular hole is greater than 400 μm according to the slider 110 when the loop 106 is the same size as the macular hole, the surgeon may proceed with an ILM flap procedure.

As discussed in greater detail below, though the loop 106 is demonstrated in FIG. 2, any end effectors capable of including a measurement component as described herein may be utilized in a similar manner to determine the size of a patient's macular hole and, as such, the type of procedure the surgeon may perform.

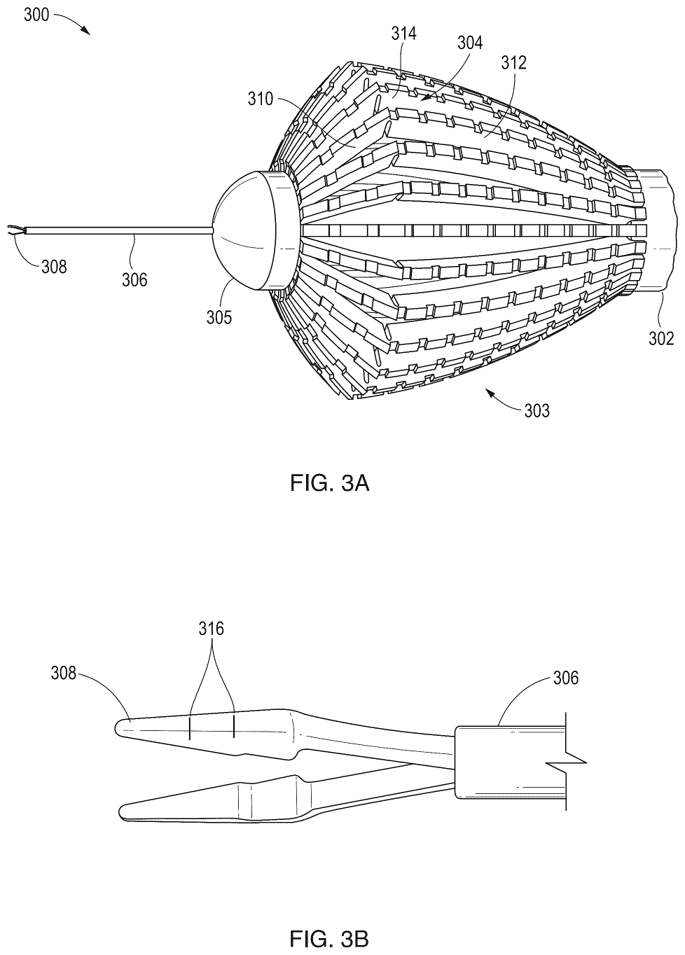

FIG. 3A is a perspective view of an exemplary surgical instrument 300 having an end effector, such as forceps 308, according to embodiments described herein. The surgical instrument 300 includes a handle 302, a basket 303 comprising a plurality of actuation levers 304, a housing 305, an outer tube 306, and an end effector, shown as forceps 308, at the tip of the surgical instrument 300. Each actuation lever 304 comprising a first leg 312 and a second leg 310 joined at flexible juncture 314. Each actuation lever may be formed of a material such as shape memory material, titanium, stainless steel, suitable thermoplastic, etc. Outer tube 306 may be any suitable medical grade tubing such as titanium, stainless steel, or suitable polymer and is sized so that forceps 308 reciprocate easily within. Forceps 308 are generally made from stainless steel or titanium, though other materials are also contemplated.

Surgical instrument 300 is designed so that in use, when the plurality of actuation levers 304 are in their relaxed state, forceps 308 protrude or extend beyond the distal end of the outer tube 306, which is coupled to the housing 305. Squeezing one or more of the actuation levers 304 causes the respective actuation lever 304 to flex at juncture 314, pushing housing 305 forward relative to the handle 302. The forward movement of housing 305 is transferred to outer tube 306, causing outer tube 306 to slide forward over a proximal portion of the jaws of the forceps 308, thereby activating forceps 308 by compressing together the jaws. By closing jaws of forceps 308, the surgeon is able to, for example, grasp and peel a tissue (e.g., ILM) within the body.

While a basket 303 is shown in FIG. 3A, other actuation mechanisms are also contemplated to translate the outer tube 306 forward over a proximal portion of the jaws of the forceps 308 to compress the jaws of the forceps 308. For example, in certain embodiments, a slider may be utilized to translate the outer tube 306 over a proximal portion of the jaws of the forceps 308, thereby decreasing a distance between opposing distal ends of the jaws of the forceps 308. The slider may be positioned above a scale on a handle, similar to the surgical instrument described with reference to FIG. 1, such that the location of the slider along the scale corresponds to a distance the outer tube is extended. The distance the outer tube is extended corresponds to the distance between opposing distal ends of the jaws of the forceps. Therefore, the surgeon may use the distance between opposing distal ends of the jaws of the forceps to determine the diameter of an ophthalmic tissue (e.g., a macular hole) by extending or retracting the outer tube via the slider until the distance between opposing distal ends of the jaws is equivalent to the diameter of the macular hole. When the distance between opposing distal ends of the jaws is equivalent to the diameter of the macular hole, the measurement on the scale corresponds to the diameter of the macular hole.

FIG. 3B illustrates a detailed view of exemplary end effector, such as forceps 308, that may be implemented in the surgical instrument 300 of FIG. 3A. As described with reference to FIG. 3A, the outer tube 306 may be configured to slide forward over the forceps 308 to compress together the jaws of the forceps 308 to grasp and peel a tissue during a surgical procedure.

In certain embodiments, the forceps 308 may include one or more markings 316 corresponding to various measurements on one or more jaws of the forceps 308. For example, the markings 316 may include two markings 316 with a distance of 400 μm as shown in FIG. 3B. In certain other embodiments, the markings 316 may include markings every 100 μm between 0 to 500 μm, such as 0 μm, 100 μm, 200 μm, 300 μm, 400 μm, and 500 μm.

The surgeon may position the forceps 308 such that the jaw of the forceps 308 having one or more markings 316 is across the macular hole. The markings 316 on the jaws of the forceps 308 may be utilized when the forceps 308 are in either an activated state or a relaxed state. In the activated state, the jaws of the forceps 308 may be compressed by movement of the outer tube 306, and in the relaxed state, the jaws of the forceps 308 may not be compressed. The surgeon, through a microscope or other visualization method, may visualize the diameter of the macular hole relative to the markings 316 on the jaws of the forceps 308 to determine the size of the macular hole and the appropriate surgical procedure to treat the macular hole.

In certain embodiments, though the end effector is shown as forceps 308 in FIG. 3B, the end effector may be a scraper or a spatula for example. In this example, the spatula or scraper may include one or more markings corresponding to various measurements. The markings may be formed on a distal most end of the end effector or along one or more sides, for example.

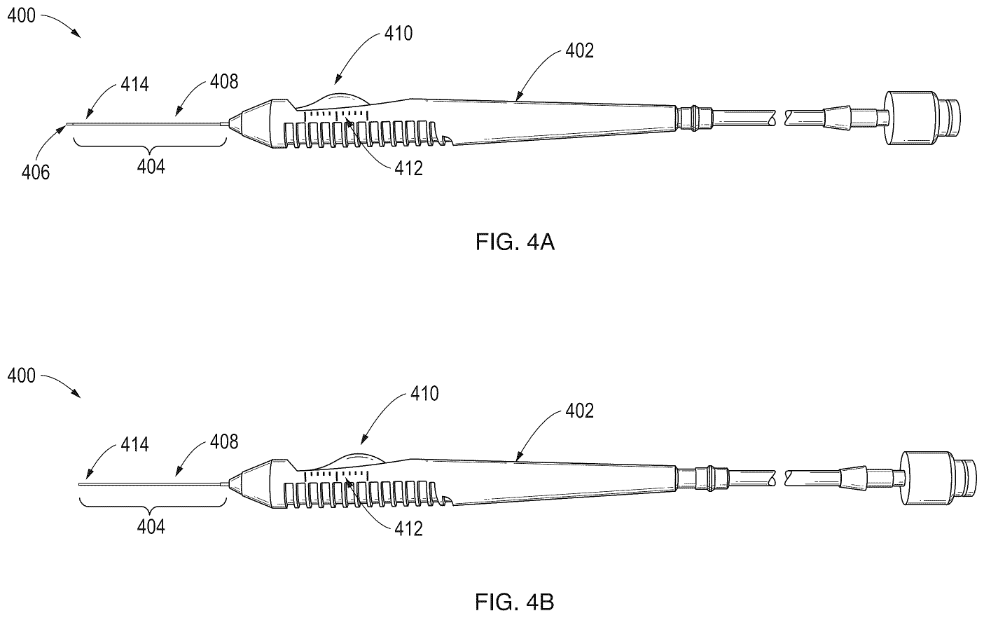

FIG. 4A illustrates a perspective view of an exemplary surgical instrument 400 having an end effector, such as a retractable soft tip 406 disposed in an extended position, according to embodiments described herein. The surgical instrument 400 includes a handle 402, an instrument portion 404, a soft tip 406 and an outer tube 408, and an actuator, such as a slider 410. Surgical instrument 400 may be configured to aspirate liquid and/or ophthalmic tissues from the eye during an ILM peeling or ILM flap procedure. Similar to surgical instrument 100, the instrument portion 404 extends from the distal end of the handle 402 and includes a soft tip 406 at the distal end 414 of the outer tube 408. The slider 410 may be a manual control structure mounted to the handle 402 and configured to translate the retractable soft tip 406 out of the distal end 414 of the outer tube 408.

The soft tip 406 may be a flexible rubber tube within the outer tube 408. In certain embodiments, as described with reference to FIG. 3B, the distal end of the soft tip 406 may include one or more markings on the rubber tube, which may be visualized by protracting the retractable soft tip 406 from the outer tube 408. For example, the markings may include two markings with a distance of 400 μm as shown in FIG. 3B. In certain other embodiments, the markings may include markings every 100 μm between 0 to 500 μm, such as 0 μm, 100 μm, 200 μm, 300 μm, 400 μm, and 500 μm.

Once the soft tip 406 is translated from the distal end 414 of the outer tube 408, the surgeon may position the soft tip 406 such that the one or more markings are positioned across the macular hole. The surgeon, through a microscope or other visualization method, may visualize the diameter of the macular hole relative to the markings on the soft tip 406 to determine the size of the macular hole and, therefore, the appropriate surgical procedure to treat the macular hole.

In certain embodiments, the surgical instrument 400 may include a scale 412 positioned on the handle 402 below the slider 410. Similar to the scale described with reference to FIG. 1, the scale 412 may include various markings at specific distances between 0 μm and 500 μm, for example. The degree of movement of the soft tip 406 out of the outer tube 408 corresponds to the scale 412 positioned on the handle 402. For example, by moving the slider 410 to the 100 μm marking on the scale 412 corresponds to the soft tip 406 protracting from the distal end 414 of the outer tube 408 a distance of 100 μm. The surgeon may release the soft tip 406 from the distal end 414 of the outer tube 408 by manually sliding the slider 410 forward until the length of the soft tip 406 matches the diameter of the macular hole. The position of the slider 410 relative to the scale 412 demonstrates the diameter of the macular hole when the length of the soft tip 406 matches the diameter of the macular hole. In some embodiments, the user may judge movement of the slider 410 relative to the scale 412 by watching movement of the highest point of the slider 410 relative to the scale 412. In some embodiments, a marking on the slider (e.g., a vertical line at the highest point of the slider) may be watched for relative movement to the scale 412.

FIG. 4B illustrates a perspective view of an exemplary surgical instrument 400 having an end effector, such as retractable soft tip 406 disposed in a retracted position, according to embodiments described herein.

As depicted in FIG. 4A, the surgical instrument 400 includes a handle 402, an instrument portion 404, a soft tip, and an outer tube 408, and an actuator, such as a slider 410. The instrument portion 404 extends from the distal end of the handle 402 and includes a soft tip housed within the distal end 414 of the outer tube 408. The slider 410 may be a manual control structure mounted to the handle 402 and configured to translate the soft tip out of the distal end 414 of the outer tube 408.

The slider 410 in FIG. 4B is shown in a retracted position, with the slider 410 translated towards the proximal end of the handle 402. In the retracted position, the soft tip is disposed completely within the outer tube 408. The surgeon may extend the soft tip from the distal end 414 of the outer tube 408 by manually sliding the slider 410 towards the distal end of the handle 402 to measure the diameter of a macular hole as described with reference to FIG. 4A.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. A surgical instrument, comprising:
a handle, comprising a distal end and a proximal end;
an instrument portion extending from the distal end of the handle, the instrument portion comprising a distal end; and
an outer tube located around the instrument portion,
wherein:
the distal end of the instrument portion comprises an end effector,
a degree of movement of the outer tube or end effector of the surgical instrument corresponds to a scale on the handle of the surgical instrument that facilitates measurement of a tissue during ophthalmic procedures, and
movement of the outer tube or the end effector facilitates measurement of the tissue.

2. The surgical instrument of claim 1, wherein the outer tube is configured to translate toward the distal end of the instrument portion upon translation of a slider.

3. The surgical instrument of claim 1, wherein the end effector of the instrument portion is a loop coupled to a slider on the handle.

4. The surgical instrument of claim 3, wherein the slider moves relative to markings on the surgical instrument corresponding to a diameter of the loop at the distal end of the instrument portion extended from the outer tube.

5. The surgical instrument of claim 1, wherein the end effector is a soft tip coupled to a slider on the handle.

6. The surgical instrument of claim 5, wherein the slider moves relative to markings on the surgical instrument corresponding to a length of the soft tip extended from the outer tube.

7. The surgical instrument of claim 1, wherein the outer tube is configured to translate toward the distal end of the end effector upon movement of a basket coupled to the handle.

8. The surgical instrument of claim 7, wherein:
the basket comprises a plurality of actuation levers having a proximal end and a distal end coupled to a tip of the basket, and
compressing one or more of the plurality of actuation levers moves the handle relative to the instrument portion and toward the end effector, causing the outer tube to translate toward the distal end of the end effector.

9. The surgical instrument of claim 1, wherein the end effector of the instrument portion is forceps.

10. The surgical instrument of claim 9, wherein a distance between a distal end of jaws of the forceps is measured by a slider on the handle and the distance between the jaws of the forceps correspond to a measurement of a macular hole.

11. An surgical instrument, comprising:
a handle, comprising a distal end and a proximal end; and
an instrument portion extending from the distal end of the handle, the instrument portion comprising a distal end,
wherein:
the distal end of the instrument portion comprises an end effector, and
the end effector has a measurement scale thereon corresponding to a sizing scale for facilitating measurement of a tissue during ophthalmic procedures.

12. The surgical instrument of claim 11, wherein the surgical instrument further comprises an outer tube located around the instrument portion, wherein movement of the outer tube activates the end effector.

13. The surgical instrument of claim 11, wherein the end effector comprises forceps.

14. The surgical instrument of claim 13, wherein the forceps comprise markings on a jaw of the forceps, the markings representing a measurement of 400 μm.

15. The surgical instrument of claim 11, wherein the end effector comprises a spatula and the spatula comprises markings corresponding to one or more measurements.
